(19) United States
(12) Patent Application Publication
(10) Pub. No.: US 2017/0193675 A1
(43) Pub. Date: Jul. 6, 2017

(54) APPARATUS AND METHOD FOR ANALYZING RADIOGRAPHY POSITION ERROR OR RADIOGRAPHY CONDITION ERROR OR BOTH BASED ON X-RAY RADIOGRAPH

(71) Applicants: VATECH Co., Ltd., Gyeonggi-do (KR); VATECH EWOO Holdings Co., Ltd., Gyeonggi-do (KR)

(72) Inventors: Tae Hee HAN, Gyeonggi-do (KR); Dong Wan SEO, Gyeonggi-do (KR); Se Yeol IM, Gyeonggi-do (KR)

(73) Assignees: VATECH Co., Ltd., Gyeonggi-do (KR); VATECH EWOO Holdings Co., Ltd., Gyeonggi-do (KR)

(21) Appl. No.: 15/397,774

(22) Filed: Jan. 4, 2017

(30) Foreign Application Priority Data
Jan. 4, 2016 (KR) 10-2016-0000416

(51) Int. Cl. G06T 7/70; A61B 6/00; G06T 7/00; A61B 6/14

(52) U.S. Cl. CPC G06T 7/70; A61B 6/14; A61B 6/5258; G06T 7/0012; G06T 2207/10116; G06T 2207/30036; A61B 6/461

(57) ABSTRACT

Disclosed is an apparatus and method for analyzing a radiography position error or a radiography condition error or both on basis of an x-ray radiograph. The apparatus includes an error data output unit outputting error data indicating that a radiography position or a radiography condition or both are incorrect by analyzing the X-ray radiograph; and an error data record unit recording the error data, wherein the error data output unit includes at least one of: a horizontal alignment error analysis unit analyzing a horizontal alignment error; a vertical alignment error analysis unit analyzing a vertical alignment error; a canine tooth alignment error analysis unit analyzing a canine tooth alignment error; an irradiation condition selection error analysis unit analyzing an irradiation condition selection error; a lead apron wearing error analysis unit analyzing a lead apron wearing error; and a Frankfort line alignment error analysis unit analyzing a Frankfort line alignment error.

### CROSS REFERENCE TO RELATED APPLICATION

The present application claims priority to Korean Patent Application No. 10-2016-0000416, filed Jan. 4, 2016, the entire contents of which is incorporated herein for all purposes by this reference.

### BACKGROUND OF THE INVENTION

Field of the Invention

The present invention relates generally to a technique of analyzing a medical image and, more particularly, to a technique of distinguishing that a position of a patient or a radiography condition is appropriate during X-ray radiography, by analyzing an X-ray radiograph.

Description of the Related Art

An X-ray radiograph is generally used for dental diagnoses and treatments. Various X-ray radiographs obtained in various ways are widely known. Among these, a two-dimensional panorama radiograph and cephalometric radiograph are advantageously used in establishing a treatment planning, straightening teeth, etc. because it is possible to check a whole oral structure and identify an anatomical reference point better by using such radiographs. However, the panorama radiograph and the cephalometric radiograph are two-dimensional images, and thus, patient position and a radiography condition should be strictly observed during X-ray radiography in comparison with a three-dimensional CT radiograph obtained by performing three-dimensional direction change. However, in practice, a patient may move and may have inaccurate positions during X-ray radiography. For example, the radiography may be performed when a patient raises or lowers his/her head, cocks the head to the left and right, and when a canine tooth alignment is not performed. For another example, when a patient incorrectly wears a lead apron, the lead apron may be present in the result radiograph by being exposed to a radiation path of X-rays. When radiographing without satisfying radiography conditions such as a proper amount of X-ray radiation, etc., obtained radiographs that are too light or too dark may cause difficulty in distinguishing teeth. Therefore, it is common that an operator of an X-ray apparatus suspects failure in the X-ray apparatus, and requests servicing of the apparatus.

The foregoing is intended merely to aid in the understanding of the background of the present invention, and is not intended to mean that the present invention falls within the purview of the related art that is already known to those skilled in the art.

### SUMMARY OF THE INVENTION

Accordingly, the present invention has been made keeping in mind the above problems occurring in the related art, and the present invention is intended to propose an apparatus and method for identifying radiography errors, namely, a patient position error or a radiography condition error or both by analyzing an obtained X-ray radiograph.

It is to be understood that problems to be solved by the present invention are not limited to the aforementioned problems and other problems that are not mentioned will be apparent from the following description to a person with an ordinary skill in the art to which the present invention pertains.

In order to achieve the above object, according to embodiments of the present invention, there is provided an apparatus for analyzing a radiography position error or a radiography condition error or both based on an X-ray radiograph, the apparatus including: an error data output unit outputting error data indicating that a radiography position or a radiography condition or both are incorrect by analyzing the X-ray radiograph; and an error data record unit recording the error data. Here, the error data output unit includes at least one of a horizontal alignment error analysis unit analyzing a horizontal alignment error based on the X-ray radiograph; a vertical alignment error analysis unit analyzing a vertical alignment error based on the X-ray radiograph; a canine tooth alignment error analysis unit analyzing a canine tooth alignment error based on the X-ray radiograph; an irradiation condition selection error analysis unit analyzing an irradiation condition selection error based on the X-ray radiograph; a lead apron wearing error analysis unit analyzing a lead apron wearing error based on the X-ray radiograph; and a Frankfort line alignment error analysis unit analyzing a Frankfort line alignment error based on the X-ray radiograph.

In an embodiment, the horizontal alignment error analysis unit may detect an occlusal surface line indicating occlusal surfaces of teeth in the X-ray radiograph, and may output the error data by comparing the detected occlusal surface line with a standard occlusal surface line.

In an embodiment, the vertical alignment error analysis unit may generate first and second jaw lines by detecting left and right jaw lines in the X-ray radiograph, and may output the error data by comparing the generated first and second jaw lines with standard left and right jaw lines, respectively.

In an embodiment, the canine tooth alignment error analysis unit may detect an area indicating a canine tooth in the X-ray radiograph, and may output the error data by comparing a size of the detected area indicating the canine tooth with a size of a standard canine tooth.

In an embodiment, the irradiation condition selection error analysis unit may output the error data based on a luminance value of the X-ray radiograph.

In an embodiment, the lead apron wearing error analysis unit may detect an area indicating a lead apron in the X-ray radiograph, and may output the error data in response to the detection.

In an embodiment, the Frankfort line alignment error analysis unit may detect a Frankfort line in the X-ray radiograph and may compare the detected Frankfort line with an imaginary horizontal line, or may detect an area between a forehead ruler and a patient's forehead and may compare the detected area between the forehead ruler and the patient's forehead with a standard area, so as to output the error data.

According to embodiments of the present invention, there is provided a method of analyzing a radiography position error or a radiography condition error or both based on an X-ray radiograph, the method including: outputting error data indicating that a radiography position or a radiography condition or both are incorrect by analyzing the X-ray radiograph; and recording the error data. Here, the outputting of the error data includes at least one of analyzing a horizontal alignment error based on the X-ray radiograph; analyzing a vertical alignment error based on the X-ray radiograph; analyzing a canine tooth alignment error based on the X-ray radiograph; analyzing an irradiation condition selection error based on the X-ray radiograph; analyzing a lead apron wearing error based on the X-ray radiograph; and analyzing a Frankfort line alignment error based on the X-ray radiograph.

In an embodiment, the analyzing of the horizontal alignment error may include: detecting an occlusal surface line indicating occlusal surfaces of teeth in the X-ray radiograph, and outputting the error data by comparing the detected occlusal surface line with a standard occlusal surface line.

In an embodiment, the analyzing of the vertical alignment error may include: generating first and second jaw lines by detecting left and right jaw lines in the X-ray radiograph, and outputting the error data by comparing the generated first and second jaw lines with standard left and right jaw lines, respectively.

In an embodiment, the analyzing of the canine tooth alignment error may include: detecting an area indicating a canine tooth in the X-ray radiograph, and outputting the error data by comparing a size of the detected area indicating the canine tooth with a size of a standard canine tooth.

In an embodiment, the analyzing of the irradiation condition selection error may include: outputting the error data based on a luminance value of the X-ray radiograph.

In an embodiment, the analyzing of the lead apron wearing error may include: detecting an area indicating a lead apron in the X-ray radiograph, and outputting the error data in response to the detection.

In an embodiment, the analyzing of the Frankfort line alignment error may include: detecting a Frankfort line in the X-ray radiograph and comparing the detected Frankfort line with an imaginary horizontal line, or detecting an area between a forehead ruler and a patient's forehead and comparing the detected area between the forehead ruler and the patient's forehead with a standard area, so as to output the error data.

According to the embodiments of the present invention, the apparatus and method can identify and record the patient position error or the radiography condition error or both in performing X-ray radiography by analyzing the obtained X-ray radiograph. Such records can be provided to the operator of the X-ray apparatus, whereby the operator can effectively train users of the apparatus. Furthermore, unnecessary requests for servicing can be prevented.

### BRIEF DESCRIPTION OF THE DRAWINGS

The above and other objects, features and other advantages of the present invention will be more clearly understood from the following detailed description when taken in conjunction with the accompanying drawings, in which:

FIG. 1 is a view showing an embodiment of an apparatus for analyzing a radiography position error or a radiography condition error or both based on an X-ray radiograph according to the present invention;

FIG. 2 is a flowchart showing an embodiment of a method of analyzing a radiography position error or a radiography condition error or both based on an X-ray radiograph according to the present invention;

FIG. 3 is a view showing a first embodiment of an X-ray radiograph that is analyzed as having a horizontal alignment error according to an embodiment of the present invention;

FIG. 4 is a view showing a second embodiment of an X-ray radiograph that is analyzed as having a horizontal alignment error according to an embodiment of the present invention;

FIG. 5 is a view showing an embodiment of an X-ray radiograph that is free of errors;

FIG. 6 is a view showing a first embodiment of an X-ray radiograph that is analyzed as having a vertical alignment error according to an embodiment of the present invention;

FIG. 7 is a view showing a second embodiment of an X-ray radiograph that is analyzed as having a vertical alignment error according to an embodiment of the present invention;

FIG. 8 is a view showing a first embodiment of an X-ray radiograph that is analyzed as having a Frankfort line alignment error according to an embodiment of the present invention; and

FIG. 9 is a view showing a second embodiment of an X-ray radiograph that is analyzed as having a Frankfort line alignment error according to an embodiment of the present invention.

### DETAILED DESCRIPTION OF THE INVENTION

The above and other aspects, features, and advantages of the present invention will become apparent from the detailed description of the following embodiments in conjunction with the accompanying drawings. It should be understood that the present invention is not limited to the following embodiments and may be embodied in different ways, and that the embodiments are given to provide complete disclosure of the invention and to provide a thorough understanding of the present invention to those skilled in the art. The scope of the present invention is defined only by the claims.

The terminology used herein is for the purpose of describing particular embodiments only and is not intended to be limiting. As used herein, the singular forms “a”, “an”, and “the” are intended to include the plural forms as well, unless the context clearly indicates otherwise. It will be further understood that the terms “comprise”, “include”, “have”, etc. when used in this specification, specify the presence of stated features, integers, steps, operations, elements, components, and/or combinations of them but do not preclude the presence or addition of one or more other features, integers, steps, operations, elements, components, and/or combinations thereof. Also, in the embodiments of the present invention, a “module” or a “unit” may mean a functional portion performing at least one function or operation.

Unless otherwise defined, all terms including technical and scientific terms used herein have the same meaning as commonly understood by one of ordinary skill in the art to which the present invention belongs. It will be further understood that terms used herein should be interpreted as having a meaning that is consistent with their meaning in the context of this specification and the relevant art and will not be interpreted in an idealized or overly formal sense unless expressly so defined herein.

Hereinafter, embodiments of the present invention will be described in detail with reference to the accompanying drawings. However, in the following description, it should be noted that, when the functions of conventional elements and the detailed description of elements related with the present invention may make the gist of the present invention unclear, a detailed description of those elements will be omitted.

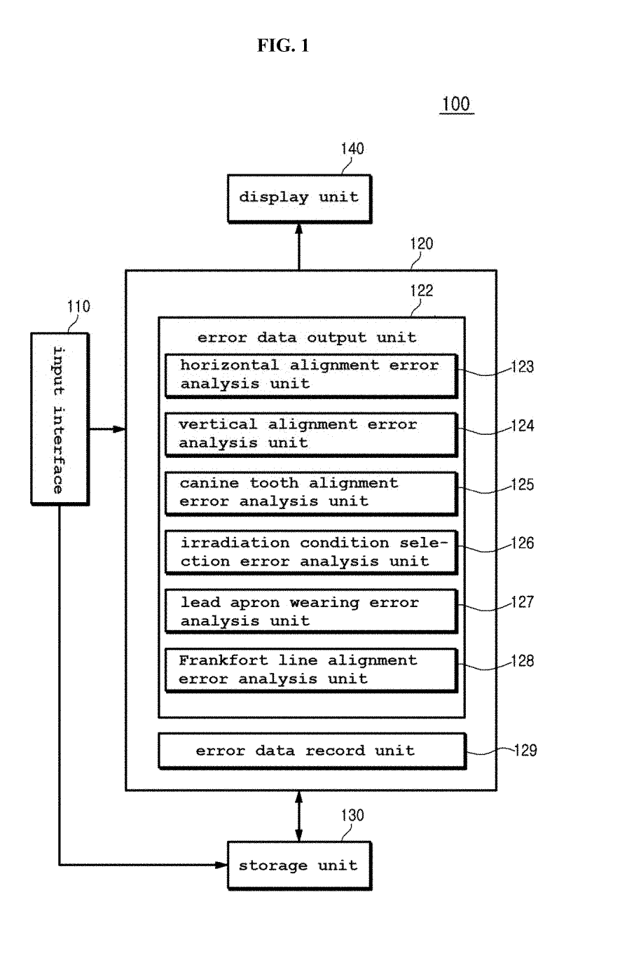

FIG. 1 is a view showing an embodiment of an apparatus for analyzing a radiography position error or a radiography condition error or both based on an X-ray radiograph according to the present invention

As shown in FIG. 1, an apparatus 100 for analyzing a radiography position error or a radiography condition error or both may include an input interface 110, a radiograph processing unit 120, a storage unit 130, and a display unit 140. The input interface 110 may be composed of hardware and software modules for inputting user commands in order to perform radiograph analyzing/processing according to various embodiments of the present invention. The input interface 110 may input various necessary commands to the radiograph processing unit 120, or may input a two-dimensional X-ray radiograph, for example, a panorama radiograph and a cephalometric radiograph, obtained by an X-ray radiography apparatus, to the storage unit 130. Alternatively, the input interface 110 may be advantageously used in directing a partial or entire area of a displayed radiograph so as to perform relevant various radiograph processing. In an embodiment, the input interface 110 may include a keyboard, a keypad, a touchpad, a mouse, etc. of a computer, but types of the input interface are not limited thereto. For example, the input interface 110 may include a graphic user interface that is controlled by using the aforementioned input devices. The display unit 140 displays radiographs obtained according to various embodiments of the present invention may include various display devices such as a liquid-crystal display (LCD), a light-emitting diode (LED) display, an active-matrix organic light-emitting diode (AMOLED) display, a cathode ray tube (CRT) display, etc.

The storage unit 130 may be used for storing a two-dimensional X-ray radiograph, for example, a panorama radiograph and a cephalometric radiograph, obtained by the X-ray radiography apparatus. The storage unit 130 may be used for storing radiograph data of an intermediate result and a final result that are obtained by performing radiograph processing according to various embodiments of the present invention, and may be used for storing variable values that are necessary to perform radiograph processing according to various embodiments of the present invention. The storage unit 130 may store a software/firmware, etc. for implementing the radiograph processing unit 120. The storage unit 130 may be implemented as one storage medium of a flash memory, a hard disk, a multimedia card (MMC), a card-type memory (for example, a secure digital (SD) card, an extreme digital (xD) card, etc.), a random access memory (RAM), a static random access memory (SRAM), a read-only memory (ROM), an electrically erasable programmable read-only memory (EEPROM), a programmable read-only memory (PROM), a magnetic memory, a magnetic disk, and an optical disk, but an implemented form of the storage unit 130 is not limited thereto.

The radiograph processing unit 120 may perform a method of analyzing a radiography position error or a radiography condition error or both based on an X-ray radiograph by deriving partial X-ray radiograph data or the entire X-ray radiograph data from the storage unit 130 according to various embodiment of the present invention. As shown in FIG. 1, the radiograph processing unit 120 may include an error data output unit 122 and an error data record unit 129. The error data output unit 122 may output error data indicating that a radiography position or a radiography condition or both are incorrect by analyzing an X-ray radiograph. The error data record unit 129 may record the error data outputted from the error data output unit 122.

The error data output unit 122 may include a horizontal alignment error analysis unit 123 analyzing a horizontal alignment error based on the X-ray radiograph; a vertical alignment error analysis unit 124 analyzing a vertical alignment error based on the X-ray radiograph; a canine tooth alignment error analysis unit 125 analyzing a canine tooth alignment error based on the X-ray radiograph; a irradiation condition selection error analysis unit 126 analyzing an irradiation condition selection error based on the X-ray radiograph; a lead apron wearing error analysis unit 127 analyzing a lead apron wearing error based on the X-ray radiograph; and a Frankfort line alignment error analysis unit 128 analyzing a Frankfort line alignment error based on the X-ray radiograph. In an embodiment of FIG. 1, the error data output unit 122 includes the horizontal alignment error analysis unit 123, the vertical alignment error analysis unit 124, the canine tooth alignment error analysis unit 125, the irradiation condition selection error analysis unit 126, the lead apron wearing error analysis unit 127, and the Frankfort line alignment error analysis unit 128 all, but the error data output unit 122 may include only one or some of the components.

For reference, the horizontal alignment error analysis unit 123 and the vertical alignment error analysis unit 124 may analyze respective errors of a panorama radiograph and a posteroanterior (PA) cephalometric radiograph, the canine tooth alignment error analysis unit 125 may analyze an error of a panorama radiograph, the irradiation condition selection error analysis unit 126 and the lead apron wearing error analysis unit 127 may analyze respective errors of a panorama radiograph and PA and lateral (LAT) cephalometric radiographs, and the Frankfort line alignment error analysis unit 128 may analyze an error of the LAT cephalometric radiograph, but the error analysis target image of each of the analysis units is not limited thereto.

The horizontal alignment error analysis unit 123 may detect an occlusal surface line indicating occlusal surfaces of teeth in the X-ray radiograph, and may output error data based on analysis of the detected occlusal surface line. In an embodiment, the horizontal alignment error analysis unit 123 may compare the detected occlusal surface line with a standard occlusal surface line. In an embodiment, the horizontal alignment error analysis unit 123 may determine whether a difference value between curvature of the detected occlusal surface line and curvature of the standard occlusal surface line is equal to or greater than a first threshold value. Here, like an average value of curvatures of occlusal surface lines in typical X-ray radiographs that are obtained after horizontal alignment, the curvature of the standard occlusal surface line may be an experimentally determined value. The first threshold value may be a statistically determined value. When the difference value between the curvature of the detected occlusal surface line and the curvature of the standard occlusal surface line is equal to or greater than the first threshold value, the horizontal alignment error analysis unit 123 may determine that an X-ray radiograph, which is an analysis target, is an X-ray radiograph obtained before horizontal alignment, and may output the error data.

The vertical alignment error analysis unit 124 may generate first and second jaw lines by detecting left and right jaw lines in the X-ray radiograph, and may output error data based on analysis of the generated first and second jaw lines. In an embodiment, the vertical alignment error analysis unit 124 may determine whether a difference between a curvature difference value of the first and second jaw lines and a curvature difference value of the standard left and right jaw lines is equal to or greater than a second threshold value. Like an average value of curvature difference values between left and right jaw lines in typical X-ray radiographs obtained after vertical alignment, the curvature difference value of the standard left and right jaw lines may be an experimentally determined value. The second threshold value may be a statistically determined value. When the difference between the curvature difference value of the first and second jaw lines and the curvature difference value of the standard left and right jaw lines is equal to or greater than the second threshold value, the vertical alignment error analysis unit 124 may determine that an X-ray radiograph, which is an analysis target, is an X-ray radiograph obtained before vertical alignment, and may output the error data.

The canine tooth alignment error analysis unit 125 may detect an area indicating a canine tooth in the X-ray radiograph, and may output error data based on analysis of the detected area indicating the canine tooth. In an embodiment, the canine tooth alignment error analysis unit 125 may determine whether a difference value between a size of the detected area indicating the canine tooth and a size of a standard canine tooth is equal to or greater than a third threshold value. Here, like an average value of sizes of canine teeth in typical X-ray radiographs obtained after canine tooth alignment, the size of the standard canine tooth may be an experimentally determined value. The third threshold value may be a statistically determined value. When the difference value between the size of the detected area indicating the canine tooth and the size of the standard canine tooth is equal to or greater than the third threshold value, the canine tooth alignment error analysis unit 125 may determine that an X-ray radiograph, which is an analysis target, is an X-ray radiograph obtained before canine tooth alignment, and may output the error data.

The irradiation condition selection error analysis unit 126 may calculate a representative luminance value of an X-ray radiograph, and may output error data based on analysis of the calculated representative luminance value. In an embodiment, the representative luminance value may be an average or a sum of luminance values of pixels of the X-ray radiograph, but the representative luminance value is not limited thereto. In an embodiment, the irradiation condition selection error analysis unit 126 may determine whether a difference value between the average or the sum and a standard irradiation condition value is equal to or greater than a fourth threshold value. Here, the standard irradiation condition value may be a value suggested by the manufacturer of the X-ray radiography apparatus. The fourth threshold value may be a statistically determined value or a deviation value suggested by the manufacturer. When the difference value between the average or the sum and the standard irradiation condition value is equal to or greater than the fourth threshold value, the irradiation condition selection error analysis unit 126 may determine that the X-ray radiograph, which is an analysis target, is an X-ray radiograph obtained without satisfying an irradiation condition due to underexposure or overexposure, and may output the error data.

The lead apron wearing error analysis unit 127 may detect an area indicating a lead apron in an X-ray radiograph, and may output error data in response to the detection.

The Frankfort line alignment error analysis unit 128 may detect a Frankfort line in an X-ray radiograph, and may output error data based on analysis of the detected Frankfort line.

To this end, the Frankfort line alignment error analysis unit 128 may determine whether an angle of the detected Frankfort line with respect to an imaginary horizontal line is equal to or greater than a fifth threshold value in the X-ray radiograph. Here, the fifth threshold value may be a statistically determined value. When the angle of the detected Frankfort line with respect to the imaginary horizontal line is equal to or greater than the fifth threshold value in the X-ray radiograph, the Frankfort line alignment error analysis unit 128 may determine that the X-ray radiograph, which is an analysis target, is an X-ray radiograph obtained before Frankfort line alignment, and may output the error data.

On the other hand, the Frankfort line alignment error analysis unit 128 may detect an area between a forehead ruler and a patient's forehead in an X-ray radiograph, and may output error data based on analysis of the detected area between the forehead ruler and the patient's forehead. To this end, the Frankfort line alignment error analysis unit 128 may determine whether a difference value between the detected area between the forehead ruler and the patient's forehead, and a standard area is equal to or greater than a fifth threshold value. Here, like an average value of areas between forehead rulers and patient foreheads in typical X-ray radiographs obtained after Frankfort line alignment, the standard area may be an experimentally determined value. The fifth threshold value may be a statistically determined value. When the difference value between the detected area between the forehead ruler and the patient's forehead, and the standard area is equal to or greater than the fifth threshold value, the Frankfort line alignment error analysis unit 128 may determine that the X-ray radiograph, which is an analysis target, is an X-ray radiograph obtained before Frankfort line alignment, and output the error data.

In terms of a hardware, the radiograph processing unit 120 may be implemented by using at least one of an application specific integrated circuit (ASIC), a digital signal processor (DSP), a digital signal processing device (DSPD), a programmable logic device (PLD), a field-programmable gate array (FPGA), a processor, a controller, a micro-controller, and a microprocessor. Also, the radiograph processing unit 120 may be implemented by a firmware/software module supported by a platform of the aforementioned hardware. In this case, the firmware/software module may be implemented by at least one software application written in a proper program language.

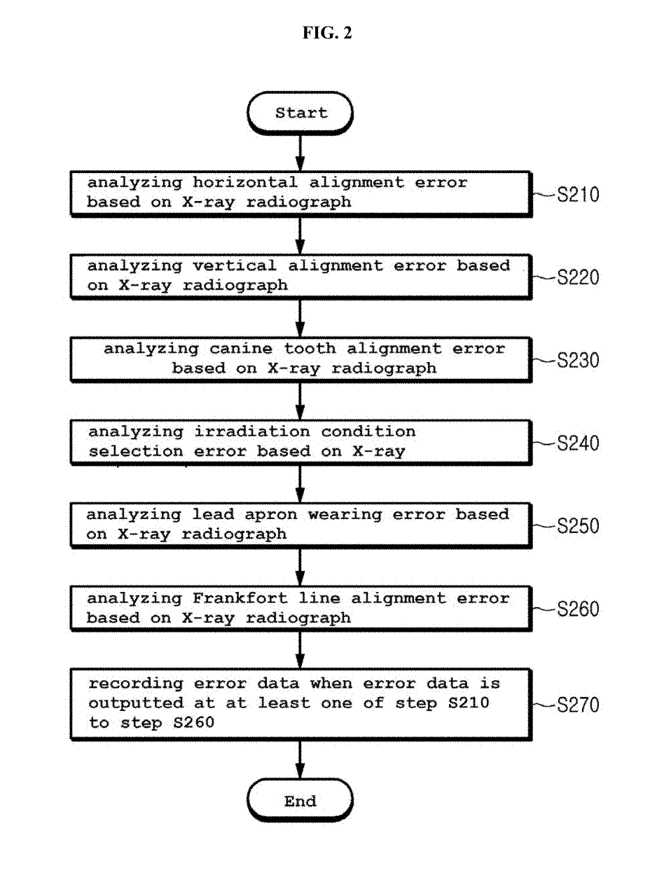

FIG. 2 is a flowchart showing an embodiment of a method of analyzing a radiography position error or a radiography condition error or both based on an X-ray radiograph according to the present invention.

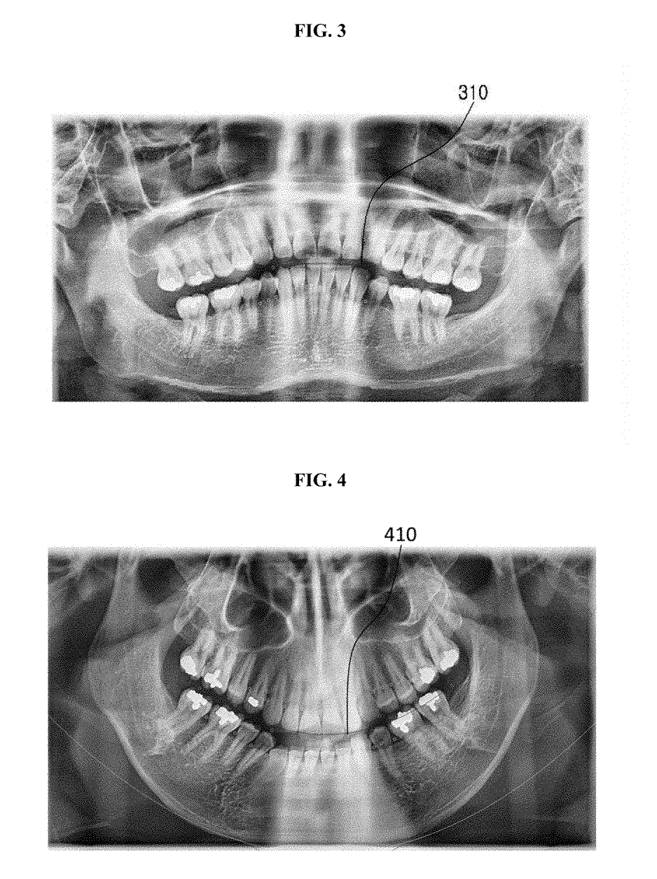

According to an embodiment of the present invention, a method of analyzing a radiography position error or a radiography condition error or both includes analyzing a horizontal alignment error based on an X-ray radiograph at step S210. At this step, the horizontal alignment error analysis unit 123 detects an occlusal surface line indicating occlusal surfaces of teeth in the X-ray radiograph, and analyses the detected occlusal surface line. In an embodiment, the occlusal surface line may be detected by generating projection data relative to a region indicating teeth and searching for valley points in the projection data. Referring to FIGS. 3 and 4 showing embodiments of X-ray radiographs that are analyzed as having horizontal alignment errors according to an embodiment of the present invention, the detected occlusal surface lines of teeth in the X-ray radiographs are designated by reference numerals 310 and 410. When the occlusal surface line is detected, the detected occlusal surface line is compared with a standard occlusal surface line. In an embodiment, whether a difference value between curvature of the detected occlusal surface line and curvature of the standard occlusal surface line is equal to or greater than the first threshold value is determined. When the difference value between the curvature of the detected occlusal surface line and the curvature of the standard occlusal surface line is equal to or greater than the first threshold value, the X-ray radiograph, which is an analysis target, may be determined as an X-ray radiograph obtained before horizontal alignment. Here, like an average value of curvatures of occlusal surface lines in typical X-ray radiographs that are obtained after horizontal alignment, the curvature of the standard occlusal surface line may be an experimentally determined value. The first threshold value may be a statistically determined value.

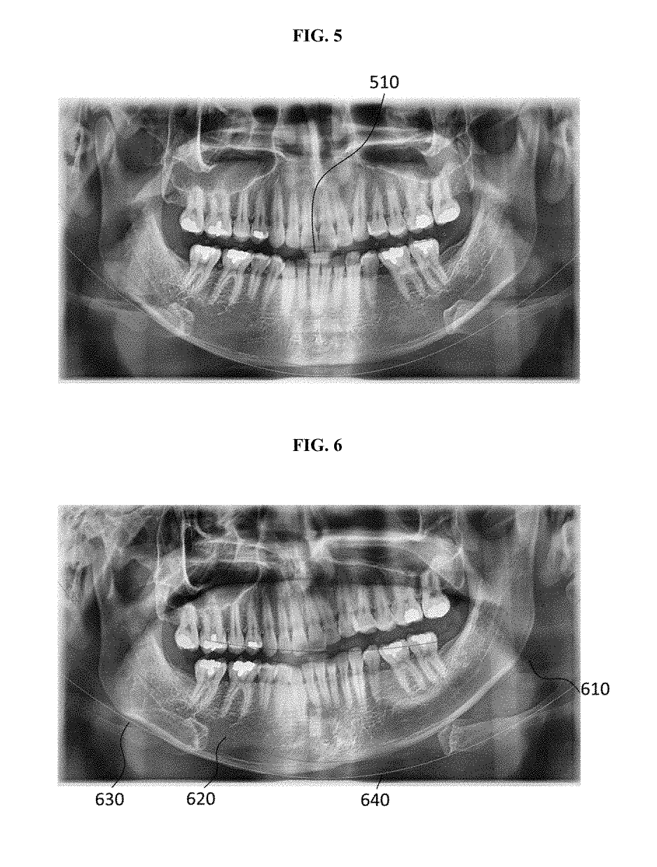

When the difference value between the curvature of the detected occlusal surface line and the curvature of the standard occlusal surface line is equal to or greater than the first threshold value, the horizontal alignment error analysis unit 123 outputs error data. In an embodiment, the error data may include an error type (horizontal alignment error), the serial number of the relevant X-ray radiograph, a date or a time or both in which the relevant X-ray radiograph is obtained, etc. Comparing FIGS. 3 and 4 with FIG. 5 showing an embodiment of an X-ray radiograph that is free of errors, an occlusal surface line 310 of FIG. 3 is excessively upwardly curved in comparison with an occlusal surface line 510 of FIG. 5, and an occlusal surface line 410 of FIG. 4 is excessively downwardly curved in comparison with an occlusal surface line 510 of FIG. 5.

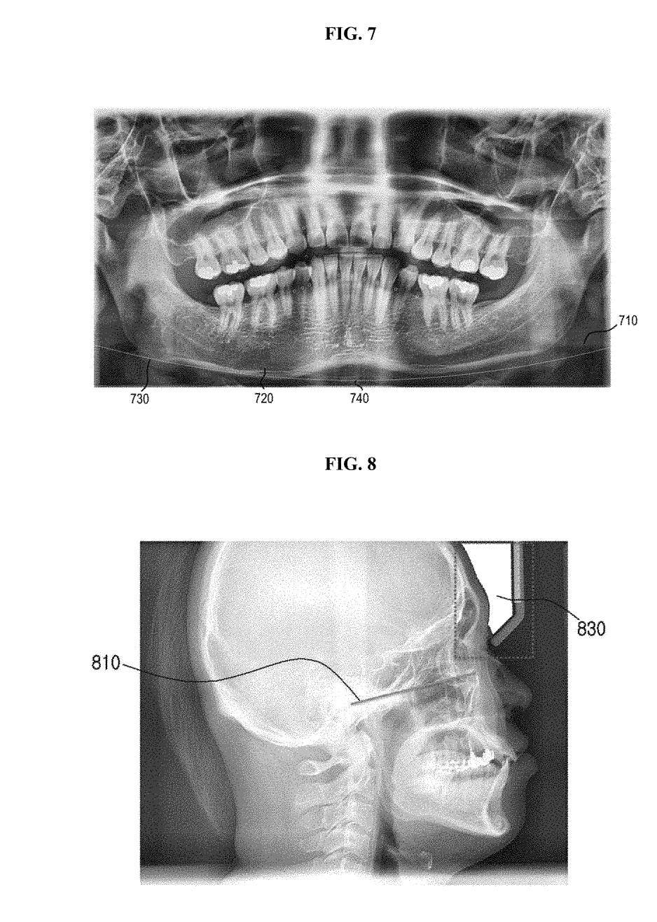

A vertical alignment error is analyzed based on an X-ray radiograph at step S220. At this step, the vertical alignment error analysis unit 124 detects left and right jaw lines in the X-ray radiograph. In an embodiment, the left and right jaw lines may be detected by using a radiograph processing technique such as a contour line detecting technique, etc. When the left jaw line is detected, a first jaw line is generated by mirroring the detected left jaw line to the right side. In an embodiment, the mirroring may be performed by using a curve-fitting algorithm such as extrapolation, etc. Referring to FIGS. 6 and 7 showing embodiments of X-ray radiographs that are analyzed as having vertical alignment errors according to an embodiment of the present invention, the detected left jaw lines are designated by reference numerals 610 and 710, and the first jaw lines are designated by reference numerals 620 and 720. Similarly, when the right jaw line is detected, a second jaw line is generated by mirroring the detected right jaw line to the left side. As described above, the mirroring may be performed by using the curve-fitting algorithm. Referring to FIGS. 6 and 7, the detected right jaw lines are designated by reference numerals 630 and 730, and the second jaw lines are designated by reference numerals 640 and 740.

Next, the vertical alignment error analysis unit 124 determines whether a difference between a curvature difference value of the first jaw line 620 and the second jaw line 640, and a curvature difference value of the standard left and right jaw lines is equal to or greater than the second threshold value, and determines whether a difference between a curvature difference value of the first jaw line 720 and the second jaw line 740, and a curvature difference value of the standard left and right jaw lines is equal to or greater than the second threshold value. Here, like an average value of curvature difference values between left and right jaw lines in typical X-ray radiographs obtained after vertical alignment, the curvature difference value of the standard left and right jaw lines may be an experimentally determined value. The second threshold value may be a statistically determined value. When the difference between the curvature difference value of the first jaw line 620 and the second jaw line 640, and the curvature difference value of the standard left and right jaw lines is equal to or greater than the second threshold value, when the difference between the curvature difference value of the first jaw line 720 and the second jaw line 740, and the curvature difference value of the standard left and right jaw lines is equal to or greater than the second threshold value, the vertical alignment error analysis unit 124 determines that the X-ray radiograph, which is an analysis target, is an X-ray radiograph obtained before vertical alignment, and outputs error data. In an embodiment, the error data may include an error type (vertical alignment error), the serial number of the relevant X-ray radiograph, a date or a time or both in which the relevant X-ray radiograph is obtained, etc. Comparing FIGS. 5 and 6 with FIG. 7, a curvature difference between the first and second jaw lines 620 and 640 of FIG. 6 is greatly larger than a curvature difference between first and second jaw lines 530 and 570 of FIG. 5, and a curvature difference between the first and second jaw lines 720 and 740 of FIG. 7 is smaller than a curvature difference between the first and second jaw lines 530 and 570 of FIG. 5.

A canine tooth alignment error is analyzed based on an X-ray radiograph at step S230. At this step, the canine tooth alignment error analysis unit 125 detects an area indicating a canine tooth in the X-ray radiograph, and outputs error data based on analysis of the detected area indicating a canine tooth. To this end, whether a difference value between a size of the detected area indicating the canine tooth and a size of a standard canine tooth is equal to or greater than the third threshold value may be determined. Here, like an average value of sizes of canine teeth in typical X-ray radiographs obtained after canine tooth alignment, the size of the standard canine tooth may be an experimentally determined value. The third threshold value may be a statistically determined value. In an embodiment, the area indicating a canine tooth in the X-ray radiograph may be distinguished through radiograph processing such as generating projection data, etc. When the difference value between the size of the detected are indicating the canine tooth and the size of the standard canine tooth is equal to or greater than the third threshold value, the canine tooth alignment error analysis unit 125 determines that the X-ray radiograph, which is an analysis target, is an X-ray radiograph obtained before canine tooth alignment, and outputs the error data. In an embodiment, the error data may include an error type (canine tooth alignment error), the serial number of the relevant X-ray radiograph, a date or a time or both in which the relevant X-ray radiograph is obtained, etc.

An irradiation condition selection error is analyzed based on an X-ray radiograph at step S240. At this step, the irradiation condition selection error analysis unit 126 calculates a representative luminance value of the X-ray radiograph, and outputs error data based on analysis of the calculated representative luminance value. In an embodiment, the representative luminance value may be an average or a sum of luminance values of pixels in the X-ray radiograph, but the representative luminance value is not limited thereto. In order to perform the analysis, the irradiation condition selection error analysis unit 126 may determine whether a difference value between the average or the sum and a standard irradiation condition value is equal to or greater than the fourth threshold value. Here, the standard irradiation condition value may be suggested by the manufacturer of the X-ray radiography apparatus. The fourth threshold value may be a statistically determined value or a deviation value suggested by the manufacturer. When the difference value between the average or the sum and the standard irradiation condition value is equal to or greater than the fourth threshold value, the irradiation condition selection error analysis unit 126 determines that the X-ray radiograph, which is an analysis target, is an X-ray radiograph obtained without satisfying an irradiation condition due to underexposure or overexposure, and outputs the error data. In an embodiment, the error data may include an error type (irradiation condition selection error), the serial number of the relevant X-ray radiograph, a date or a time or both in which the relevant X-ray radiograph is obtained, etc.

A lead apron wearing error is analyzed based on an X-ray radiograph at step S250. At this step, the lead apron wearing error analysis unit 127 detects an area indicating a lead apron in the X-ray radiograph, and outputs error data in response to the detection. In an embodiment, when an area (cluster) having a dark luminance value occurs at a lower part of the X-ray radiograph and the area is equal to or greater than a predetermined value, the area may be determined as a result of radiographing a patient incorrectly wearing the lead apron. In an embodiment, the error data may include an error type (lead apron wearing error), the serial number of the relevant X-ray radiograph, a date or a time or both in which the relevant X-ray radiograph is obtained, etc.

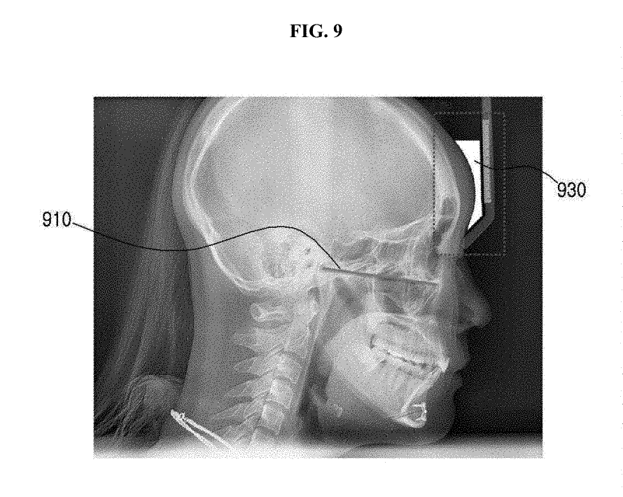

A Frankfort line alignment error is analyzed based on an X-ray radiograph at step S260. At this step, the Frankfort line alignment error analysis unit 128 detects a Frankfort line in the X-ray radiograph, and outputs error data based on analysis of the detected Frankfort line. To this end, the Frankfort line alignment error analysis unit 128 may determine whether an angle of the detected Frankfort line with respect to an imaginary horizontal line is equal to or greater than the fifth threshold value. Here, the fifth threshold value may be a statistically determined value. When the angle of the detected Frankfort line with respect to the imaginary horizontal line in the X-ray radiograph is equal to or greater than the fifth threshold value, the Frankfort line alignment error analysis unit 128 may determine that the X-ray radiograph, which is an analysis target, is an X-ray radiograph obtained before Frankfort line alignment. Referring to FIGS. 8 and 9 showing embodiments of X-ray radiographs that are analyzed as having Frankfort line alignment errors according to an embodiment of the present invention, a Frankfort line 810 of FIG. 8 is inclined upwardly with respect to the imaginary horizontal line, which means the relevant X-ray radiograph was obtained when a patient raised his/her head. In contrast, a

Frankfort line 910 of FIG. 9 is inclined downwardly with respect to the imaginary horizontal line, which means the relevant X-ray radiograph was obtained when a patient lowered his/her head. The X-ray radiographs of the both cases are X-ray radiographs obtained before Frankfort line alignment.

Alternatively, at step S260, the Frankfort line alignment error analysis unit 128 may detect an area between a forehead ruler and a patient's forehead in an X-ray radiograph, and may output error data based on analysis of the detected area between the forehead ruler and the patient's forehead. To this end, the Frankfort line alignment error analysis unit 128 may determine whether a difference value between the detected area between the forehead ruler and the patient's forehead, and a standard area is equal to or greater than the fifth threshold value. Here, like an average value of areas between forehead rulers and patient foreheads in typical X-ray radiographs obtained after Frankfort line alignment, the standard area may be an experimentally determined value. The fifth threshold value may be a statistically determined value. When the difference value between the detected area between the forehead ruler and the patient's forehead, and the standard area is equal to or greater than the fifth threshold value, the Frankfort line alignment error analysis unit 128 may determine that the X-ray radiograph, which is an analysis target, is an X-ray radiograph obtained before Frankfort line alignment, and may output the error data. In an embodiment, the error data may include an error type (Frankfort line alignment error), the serial number of the relevant X-ray radiograph, a date or a time or both in which the relevant X-ray radiograph is obtained, etc. Referring to FIGS. 8 and 9, an area 830 between a forehead ruler and a patient's forehead of FIG. 8 is larger than the standard area, and an area 930 between a forehead ruler and a patient's forehead is smaller than the standard area. The X-ray radiographs of the both cases are X-ray radiographs obtained before Frankfort line alignment.

Through step S210 to step S260, The present invention has been described about distinguishing whether or not a patient has a proper position, whether or not a patient correctly wears a lead apron, and whether or not a radiography condition selection is wrong during X-ray radiography, from an obtained X-ray radiograph, but it is unnecessary to perform steps S210 to step S260 in order as described above. It will be understood by the skilled person that step S210 to step S260 may be performed in random order within the spirit of the present invention. In addition, it is unnecessary to perform all of step S210 to step S260. It should be noted that the scope of the present invention includes a case of performing at least one of step S210 to step S260.

Next, when error data is output at at least one of step S210 to step S260, the error data record unit 129 records the error data in the storage unit 130 at step S270. The error data may include horizontal alignment error data, vertical alignment error data, canine tooth alignment error data, irradiation condition selection error data, lead apron wearing error data, and Frankfort line alignment error data. As described above, each error data may include the serial number of an X-ray radiograph in which the relevant error is detected, a date or a time or both in which the relevant X-ray radiograph is obtained, etc. An operator of the X-ray apparatus may identify which error caused a particular X-ray radiograph to be wrongly radiographed at a particular date/time by using the recorded error data.

In the described embodiments of the present invention, arrangement of the described components may vary depending on the environment or requirements to be implemented. For example, some components may be omitted, or several components may be integrated to be operated as one component. In addition, sequence of arrangement of and connection between some components may be changed.

While various embodiment of the present invention have been shown and described, the present invention is not limited to the aforementioned particular embodiments, various modifications are possible by those skilled in the art without departing from the sprit and scope of the accompanying claims, and these modifications should not be understood separately from the scope and spirit. Accordingly, the technical scope of the present invention should be defined only by the accompanying claims.

What is claimed is:

1. An apparatus for analyzing a radiography position error or a radiography condition error or both based on an X-ray radiograph, the apparatus comprising:
an error data output unit outputting error data indicating that a radiography position or a radiography condition or both are incorrect by analyzing the X-ray radiograph; and
an error data record unit recording the error data,
wherein the error data output unit comprises at least one of:
a horizontal alignment error analysis unit analyzing a horizontal alignment error based on the X-ray radiograph;
a vertical alignment error analysis unit analyzing a vertical alignment error based on the X-ray radiograph;
a canine tooth alignment error analysis unit analyzing a canine tooth alignment error based on the X-ray radiograph;
an irradiation condition selection error analysis unit analyzing an irradiation condition selection error based on the X-ray radiograph;
a lead apron wearing error analysis unit analyzing a lead apron wearing error based on the X-ray radiograph; and
a Frankfort line alignment error analysis unit analyzing a Frankfort line alignment error based on the X-ray radiograph.

2. The apparatus of claim 1, wherein the horizontal alignment error analysis unit detects an occlusal surface line indicating occlusal surfaces of teeth in the X-ray radiograph, and outputs the error data by comparing the detected occlusal surface line with a standard occlusal surface line.

3. The apparatus of claim 1, wherein the vertical alignment error analysis unit generates first and second jaw lines by detecting left and right jaw lines in the X-ray radiograph, and outputs the error data by comparing the generated first and second jaw lines with standard left and right jaw lines respectively.

4. The apparatus of claim 1, wherein the canine tooth alignment error analysis unit detects an area indicating a canine tooth in the X-ray radiograph, and outputs the error data by comparing a size of the detected area indicating the canine tooth with a size of a standard canine tooth.

5. The apparatus of claim 1, wherein the irradiation condition selection error analysis unit outputs the error data based on a luminance value of the X-ray radiograph.

6. The apparatus of claim 1, wherein the lead apron wearing error analysis unit detects an area indicating a lead apron in the X-ray radiograph, and outputs the error data in response to the detection.

7. The apparatus of claim 1, wherein the Frankfort line alignment error analysis unit detects a Frankfort line in the X-ray radiograph and compares the detected Frankfort line with an imaginary horizontal line, or detects an area between a forehead ruler and a patient's forehead and compares the detected area between the forehead ruler and the patient's forehead with a standard area, so as to output the error data.

8. A method of analyzing a radiography position error or a radiography condition error or both based on an X-ray radiograph, the method comprising:
outputting error data indicating that a radiography position or a radiography condition or both are incorrect by analyzing the X-ray radiograph; and
recording the error data,
wherein the outputting of the error data comprises at least one of:
analyzing a horizontal alignment error based on the X-ray radiograph;
analyzing a vertical alignment error based on the X-ray radiograph;
analyzing a canine tooth alignment error based on the X-ray radiograph;
analyzing an irradiation condition selection error based on the X-ray radiograph;
analyzing a lead apron wearing error based on the X-ray radiograph; and
analyzing a Frankfort line alignment error based on the X-ray radiograph.

9. The method of claim 8, wherein the analyzing of the horizontal alignment error comprises:
detecting an occlusal surface line indicating occlusal surfaces of teeth in the X-ray radiograph, and outputting the error data by comparing the detected occlusal surface line with a standard occlusal surface line.

10. The method of claim 8, wherein the analyzing of the vertical alignment error comprises:
generating first and second jaw lines by detecting left and right jaw lines in the X-ray radiograph, and outputting the error data by comparing the generated first and second jaw lines with standard left and right jaw lines respectively.

11. The method of claim 8, wherein the analyzing of the canine tooth alignment error comprises:
detecting an area indicating a canine tooth in the X-ray radiograph, and outputting the error data by comparing a size of the detected area indicating the canine tooth with a size of a standard canine tooth.

12. The method of claim 8, wherein the analyzing of the irradiation condition selection error comprises:
outputting the error data based on a luminance value of the X-ray radiograph.

13. The method of claim 8, wherein the analyzing of the lead apron wearing error comprises:
detecting an area indicating a lead apron in the X-ray radiograph, and outputting the error data in response to the detection.

14. The method of claim 8, wherein the analyzing of the Frankfort line alignment error comprises:
detecting a Frankfort line in the X-ray radiograph and comparing the detected Frankfort line with an imaginary horizontal line, or detecting an area between a forehead ruler and a patient's forehead and comparing the detected area between the forehead ruler and the patient's forehead with a standard area, so as to output the error data.
